(19) United States
(12) Patent Application Publication
(10) Pub. No.: US 2008/0306314 A1
(43) Pub. Date: Dec. 11, 2008

(54) Hydrocarbyl- and Hydroxy-Substituted Aromatic Condensate

(75) Inventors: David J. Moreton, Derbyshire (GB); Mark F. Wilkes, Derby (GB); Mark Davies, Derby (GB)

Correspondence Address: THE LUBRIZOL CORPORATION;ATTN: DOCKET CLERK, PATENT DEPT., 29400 LAKELAND BLVD., WICKLIFFE, OH, 44092, US

(73) Assignee: The Lubrizol Corporation, Wickliffe, OH (US)

(21) Appl. No.: 11/914,961

(22) PCT Filed: May 17, 2006
(86) PCT No.: PCT/US2006/019062

§ 371 Date: Jun. 9, 2008

Related U.S. Application Data
(60) Provisional application No. 60/685,097, filed on May 26, 2005.

(51) Int. Cl. C07C 13/00

(52) U.S. Cl. 585/27

(57) ABSTRACT

This invention relates to a composition containing: (a) a hydrocarbon fluid; and (b) a hydrocarbyl- and hydroxy-substituted aromatic condensate comprising the reaction product of: (1) at least 75 mole percent based on the total amount of (b)(1) and (b)(2) of a hydrocarbyl- and hydroxy-substituted aromatic compound wherein the hydrocarbyl substituent is a linear substituent containing 8 to 36 carbon atoms; (2) 0.001 to 25 mole percent based on the total amount of (b)(1) and (b)(2) of a hydrocarbyl- and hydroxy-substituted aromatic compound wherein the hydrocarbyl substituent is a branched substituent containing 8 to 20 carbon atoms; and (3) an amount of an aldehyde, ketone, or reactive equivalent thereof effective to condense (1) and (2) to form the hydro-carbyl- and hydroxy-substituted aromatic condensate. The invention further relates to method of preparing the condensate and its use as an additive in a crude oil, a liquid fuel or an oil of lubricating viscosity.

### FIELD OF INVENTION

The present invention relates to a composition comprising a hydrocarbyl- and hydroxy-substituted aromatic condensate with linear and branched hydrocarbyl groups. The invention further relates to method of preparing the condensate and its use as an additive in a hydrocarbon fluid, such as, a crude oil, a liquid fuel or an oil of lubricating viscosity.

### BACKGROUND OF THE INVENTION

It is known that additives may be added to a hydrocarbon fluid such as a crude oil, an oil of lubricating viscosity or a liquid fuel to improve low temperature performance. However, at low temperatures, for instance in an offshore pipeline, heavy paraffinic fractions in crude oil have a tendency to become waxy and eventually become crystalline and form waxy deposits or gels. Consequently, the waxy deposits constrict the pipeline causing pipes to eventually block. This reduces production rates such that it becomes necessary to chemically or mechanically remove the deposits, resulting in loss of production, down-time and increased engineering costs. Common chemical treatments include wax crystal modifiers and/or linear or branched alkyl phenol formaldehyde resins. However, commercial alkyl phenol formaldehyde resin flow improver formulations tend to gel at low temperatures, leading to blockage, even at low actives concentrations in solvent.

U.S. Pat. No. 5,039,437 discloses additives for improving low temperature flow properties of hydrocarbon oils which are derived from the condensation product of an alkylated phenol and an aldehyde. The alkyl group of the alkylated phenol is essentially linear and has between 6 and 50 carbon atoms, with the average being between 12 and 26. Further the alkyl group contains not more than 10% of alkyl groups below 12 carbon atoms; and not more than 10% of the alkyl groups with more than 26 carbon atoms.

US Patent Application 2004/0020106 discloses a jet fuel comprising a substantially branched alkyl phenol formaldehyde condensate. The branched alkyl group preferably contains 8 to 18 carbon atoms.

US Patent Application 2003/0136046 discloses a fuel composition containing at least one alkyl phenol formaldehyde condensate. The alkyl group may be linear or branched and preferably contains 8 to 18 carbon atoms.

It would be advantageous to have a composition suitable for providing at least one of acceptable low temperature properties and/or a composition with a higher actives content (i.e. reduced amount of a solvent/carrier medium), in a hydrocarbon fluid without forming unacceptable amounts of waxy deposits and/or gels. The present invention provides such a composition.

### SUMMARY OF THE INVENTION

In one embodiment the present invention provides a composition comprising:

(a) a hydrocarbon fluid; and

(b) a hydrocarbyl- and hydroxy-substituted aromatic condensate comprising the reaction product of:
(1) at least 75 mole percent based on the total amount of (b)(1) and (b)(2) of a hydrocarbyl- and hydroxy-substituted aromatic compound wherein the hydrocarbyl substituent is a linear substituent containing 8 to 36 carbon atoms;
(2) 0.001 to 25 mole percent based on the total amount of (b)(1) and (b)(2) of a hydrocarbyl- and hydroxy-substituted aromatic compound wherein the hydrocarbyl substituent is a branched substituent containing 8 to 20 carbon atoms; and
(3) an amount of an aldehyde, ketone, or reactive equivalent thereof effective to condense (1) and (2) to form the hydrocarbyl- and hydroxy-substituted aromatic condensate.

In one embodiment the invention provides a method of controlling waxy deposit and/or gel formation in a hydrocarbon fluid, the method comprising mixing/blending:

(a) a hydrocarbon fluid with:

(b) a hydrocarbyl- and hydroxy-substituted aromatic condensate comprising the reaction product of:
(1) at least 75 mole percent based on the total amount of (b)(1) and (b)(2) of a hydrocarbyl- and hydroxy-substituted aromatic compound wherein the hydrocarbyl substituent is a linear substituent containing 8 to 36 carbon atoms;
(2) 0.001 to 25 mole percent based on the total amount of (b)(1) and (b)(2) of a hydrocarbyl- and hydroxy-substituted aromatic compound wherein the hydrocarbyl substituent is a branched substituent containing 8 to 20 carbon atoms; and
(3) an amount of an aldehyde, ketone, or reactive equivalent thereof effective to condense (1) and (2) to form the hydrocarbyl- and hydroxy-substituted aromatic condensate.

### DETAILED DESCRIPTION OF THE INVENTION

The present invention provides a composition and method as described above.

### Hydrocarbyl- and Hydroxy-Substituted Aromatic Condensate

The hydrocarbyl- and hydroxy-substituted aromatic condensate in one embodiment is a hydrocarbyl-substituted phenol and in another embodiment hydrocarbyl-substituted cresol. In one embodiment the hydrocarbyl-substituted phenol has the hydrocarbyl substituent in the para position relative to the hydroxyl group of the phenol.

The hydrocarbyl- and hydroxy-substituted aromatic condensate may contain one or more of (b)(1) and one or more of (b)(2). Typically the hydrocarbyl- and hydroxy-substituted aromatic condensate contains 1 to 5 or 10 units of (b)(1) and 1 to 3 or 7 units of (b)(2).

In several embodiments the hydrocarbyl- and hydroxy-substituted aromatic condensate is a polymer with a number average molecular ranging from 500 to 50,000, 800 to 35,000, 3000 to 25,000 or 4000 to 15,000.

In several embodiments the hydrocarbyl- and hydroxy-substituted aromatic compound of (b)(1) has a linear hydrocarbyl substituent containing 12 to 36 carbon atoms, 20 to 30 carbon atoms or 24 to 28 carbon atoms.

The amount of hydrocarbyl- and hydroxy-substituted aromatic compound of (b)(1) present in several embodiments ranges from 75 mol % to 95 mol % or 80 mol % to 90 mol % based on the total amount of (1) and (2).

In several embodiments the hydrocarbyl- and hydroxy-substituted aromatic compound of (b)(2) has a branched hydrocarbyl substituent containing 8 to 20 carbon atoms, 10 to 18 carbon atoms or 10 to 15 carbon atoms.

Examples of a suitable branched alkyl phenol include iso-nonyl phenol, iso-decyl phenol, iso-dodecyl phenol or mixtures thereof.

The amount of hydrocarbyl- and hydroxy-substituted aromatic compound of (b)(2) present in several embodiments ranges from 5 mol % to 25 mol % or 10 mol % to 20 mol % based on the total amount of (1) and (2).

Typically the combined average number of carbons on the hydrocarbyl substituents of the hydrocarbyl- and hydroxy-substituted aromatic condensate is 22 to 26.

The hydrocarbyl- and hydroxy-substituted aromatic compound of (b)(1) and (b)(2) are condensed by reaction in the presence of aldehyde, ketone, or reactive equivalent thereof effective forming the hydrocarbyl- and hydroxy-substituted aromatic condensate.

Suitable aldehyde or ketone compounds used to condense (b)(1) and (b)(2) may have 1 to 10, 1 to 4 or 1 to 2 carbon atoms. In an embodiment of the invention the aldehyde is formaldehyde or a reactive equivalent thereof including formalin, paraformaldehyde or mixtures thereof.

The amount of aldehyde, ketone, or reactive equivalent thereof present to condense (b)(1) and (b)(2) typically ranges from 50 mol % to 200 mol %, 75 mol % to 150 mol % or 80 mol % to 120 mol % of the hydrocarbyl- and hydroxy-substituted aromatic condensate.

In one embodiment the hydrocarbyl- and hydroxy-substituted aromatic condensate is represented by the formula:

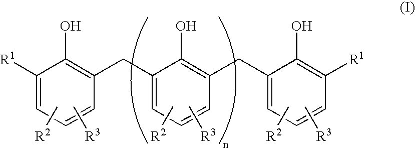

wherein

n is an integer from 0 to 10, or 1 to 8;

R¹ is hydrogen or alkyl with 1 to 4, 1 to 2 carbon atoms, or methyl;

R² is hydrogen or a linear alkyl or linear alkoxy group containing 24 to 28 carbon atoms, in one embodiment R² is in the para position relative to the hydroxyl group;

R³ is hydrogen or a branched alkyl group containing 12 to 14 carbon atoms, in one embodiment R³ is in the para position relative to the hydroxyl group; with the proviso that when R² is hydrogen, R³ is the branched alkyl group; and when R³ is hydrogen, R² is the linear alkyl or linear alkoxy group, wherein the hydrocarbyl- and hydroxy-substituted aromatic condensate comprises a hydrocarbyl-R² group substituted- and hydroxy-substituted aromatic compound which is present in an amount from 75 mol % to 95 mol % based on the total amount of (b)(1) and (b)(2); and a hydrocarbyl-R³ group substituted- and hydroxy-substituted aromatic compound which is present in an amount from 5 mol % to 25 mol % based on the total amount of (b)(1) and (b)(2).

The hydrocarbyl- and hydroxy-substituted aromatic condensate may be prepared by a process comprising reacting:
(1) at least 75 mole percent based on the total amount of (b)(1) and (b)(2) of a hydrocarbyl- and hydroxy-substituted aromatic compound wherein the hydrocarbyl substituent is a linear substituent containing 8 to 36 carbon atoms;
(2) 0.001 to 25 mole percent based on the total amount of (b)(1) and (b)(2) of a hydrocarbyl- and hydroxy-substituted aromatic compound wherein the hydrocarbyl substituent is a branched substituent containing 8 to 20 carbon atoms; and
(3) an amount of an aldehyde, ketone, or reactive equivalent thereof effective to condense (1) and (2) to form the hydrocarbyl- and hydroxy-substituted aromatic condensate.

The hydrocarbyl- and hydroxy-substituted aromatic condensate may be prepared at a reaction temperature ranging from 50° C. to 200° C., 60 C to 175° C., or 75° C. to 140° C.; the total reaction time may be from 1 minute to 24 hours, 1 hour to 18 hours or 2 hours to 12 hours. In one embodiment the reaction is carried out under reduced pressure or in an inert atmosphere, such as under nitrogen atmosphere including with a nitrogen sparge.

The hydrocarbyl- and hydroxy-substituted aromatic condensate, may also be prepared by standard techniques used to prepare an alkyl phenol condensate as described in EP 0311452 (sec for example, the techniques employed in Examples 1 to 22).

### Hydrocarbon Fluid

In one embodiment the composition contains a hydrocarbon fluid is an oil, including a crude oil or a refined oil, such as a liquid fuel, an oil of lubricating viscosity, or mixtures thereof. The hydrocarbon fluid may include synthetic liquid fuel, a synthetic oil of lubricating viscosity, or mixtures thereof. In one embodiment the hydrocarbon fluid is a crude oil. In other embodiments the hydrocarbon fluid is a liquid fuel or an oil of lubricating viscosity. The hydrocarbon fluid may be aliphatic or liquid aromatic.

### Crude Oil

The hydrocarbon fluid may be a waxy crude, or another crude prone to wax depositions, a crude, black oil or a non-volatile fraction from a distillation of a crude oil. The hydrocarbon may also be a heavy fuel such as a heavy distillate heating oil or marine/industrial fuel oil, including bunker fuel. The hydrocarbon may also be any petrochemical process oil which may form asphaltenic and ultimately coke-like species at surfaces under high temperature conditions. In one embodiment the hydrocarbon fluid is typically an oil field product, e.g., a whole well product or a multiphase mixture in or from a well bore or one at a well head after at least partial separation of gas and/or water, for instance, an oil export fraction. In one embodiment the hydrocarbon fluid is typically a refinery or petrochemical process stream or a heavy distillate or residual fuel.

### Liquid Fuel

The fuel composition of the present invention optionally comprises a liquid fuel and is useful in fueling an internal combustion engine. The liquid fuel is normally a liquid at ambient conditions. The liquid fuel may be a hydrocarbon fuel, a nonhydrocarbon fuel, or a mixture thereof. The hydrocarbon fuel may be a petroleum distillate to include a gasoline as defined by ASTM specification D4814 or a diesel fuel as defined by ASTM specification D975. In an embodiment of the invention the liquid fuel is a gasoline, and in other embodiments the liquid fuel is a leaded gasoline, or a nonleaded gasoline. In another embodiment of this invention the liquid fuel is a diesel fuel. The hydrocarbon fuel may be a hydrocarbon prepared by a gas to liquid process to include for example hydrocarbons prepared by a process such as the Fischer-Tropsch process. The nonhydrocarbon fuel may be an oxygen containing composition, often referred to as an oxygenate, to include an alcohol, an ether, a ketone, an ester of a carboxylic acid, a nitroalkane, or a mixture thereof. The nonhydrocarbon fuel may include for example methanol, ethanol, methyl t-butyl ether, methyl ethyl ketone, transesterified oils and/or fats from plants and animals such as rapeseed methyl ester and soybean methyl ester, and nitromethane. Mixtures of hydrocarbon and nonhydrocarbon fuels may include for example gasoline and methanol and/or ethanol, diesel fuel and ethanol, and diesel fuel and a transesterified plant oil such as rapeseed methyl ester. In an embodiment of the invention the liquid fuel is an emulsion of water in a hydrocarbon fuel, a nonhydrocarbon fuel, or a mixture thereof. In several embodiments of this invention the liquid fuel may have a sulphur content on a weight basis that is 5000 ppm or less, 1000 ppm or less, 300 ppm or less, 200 ppm or less, 30 ppm or less, or 10 ppm or less.

### Oil of Lubricating Viscosity

The oil of lubricating viscosity includes natural or synthetic oils of lubricating viscosity, oil derived from hydrocracking, hydrogenation, hydrofinishing, unrefined, refined and re-refined oils, or mixtures thereof. In one embodiment the oil of lubricating viscosity is a carrier fluid for the dispersant and/or other performance additives.

Natural oils include animal oils, vegetable oils, mineral oils or mixtures thereof. Synthetic oils include a hydrocarbon oil, a silicon-based oil, a liquid ester of phosphorus-containing acid. Synthetic oils may be produced by Fischer-Tropsch reactions and typically may be hydroisomerised Fischer-Tropsch hydrocarbons or waxes.

Oils of lubricating viscosity may also be defined as specified in the American Petroleum Institute (API) Base Oil Interchangeability Guidelines. In one embodiment the oil of lubricating viscosity comprises an API Group I, II, III, IV, V or mixtures thereof, and in another embodiment API Group I, II, III or mixtures thereof.

If present, the amount of an oil of lubricating viscosity and/or a liquid fuel varies in several embodiments from 35 wt % to 99.9 wt % of the composition or 50 wt % to 99.8 wt % of the composition.

### Additional Performance Additive

The composition optionally comprises one or more additional performance additives. The other performance additives include metal deactivators, detergents, dispersants, viscosity modifiers, friction modifiers, dispersant viscosity modifiers, extreme pressure agents, antiwear agents, antioxidants, corrosion inhibitors, foam inhibitors, demulsifiers, pour point depressants, seal swelling agents, wax control polymers, scale inhibitors, gas-hydrate inhibitors and mixtures thereof.

The total combined amount of the additional performance additive compounds present on an oil free basis ranges from 0 wt % to 25 wt % or 0.01 wt % to 20 wt % of the composition. Although one or more of the other performance additives may be present, it is common for the other performance additives to be present in different amounts relative to each other.

In one embodiment the composition is in a concentrate forming amount. If the present invention may be in the form of a concentrate (which may be combined with additional oil to form, in whole or in part, a finished lubricant and/or liquid fuel), the ratio of the additive of the invention and/or other additional performance additives in an oil of lubricating viscosity and/or liquid fuel, to diluent oil is in the range of 80:20 to 10:90 by weight.

Antioxidants include molybdenum dithiocarbamates, sulphurised olefins, hindered phenols, diphenylamines; detergents include neutral or overbased, Newtonian or non-Newtonian, basic salts of alkali, alkaline earth and transition metals with one or more of phenates, sulphurised phenates, sulphonates, carboxylic acids, phosphorus acids, mono- and/or di-thiophosphoric acids, saligenins, an alkylsalicylates, salixarates. Dispersants include N-substituted long chain alkenyl succinimide as well as posted treated version thereof, post-treated dispersants include those by reaction with urea, thiourea, dimercaptothiadiazoles, carbon disulphide, aldehydes, ketones, carboxylic acids, hydrocarbon-substituted succinic anhydrides, nitriles, epoxides, boron compounds, and phosphorus compounds.

Antiwear agents include compounds such as metal thiophosphates, especially zinc dialkyldithiophosphates; phosphoric acid esters or salt thereof; phosphites; and phosphorus-containing carboxylic esters, ethers, and amides. Antiscuffing agents including organic sulphides and polysulphides, such as benzyldisulphide, bis-(chlorobenzyl)disulphide, dibutyl tetrasulphide, di-tertiary butyl polysulphide, di-tert-butylsulphide, sulphurised Diels-Alder adducts or alkyl sulphenyl N′N-dialkyl dithiocarbamates. Extreme Pressure (EP) agents including chlorinated wax, organic sulphides and polysulphides, such as benzyldisulphide, bis-(chlorobenzyl)disulphide, dibutyl tetrasulphide, sulphurised methyl ester of oleic acid, sulphurised alkylphenol, sulphurised dipentene, sulphurised terpene, and sulphurised Diels-Alder adducts; phosphosulphurised hydrocarbons, metal thiocarbamates, such as zinc dioctyldithiocarbamate and barium heptylphenol diacid.

Friction modifiers include fatty amines, esters such as borated glycerol esters, partial esters of glycerol such as glycerol monooleate, fatty phosphites, fatty acid amides, fatty epoxides, borated fatty epoxides, alkoxylated fatty amines, borated alkoxylated fatty amines, metal salts of fatty acids, fatty imidazolines, condensation products of carboxylic acids and polyalkylene-polyamines, amine salts of alkylphosphoric acids. Viscosity modifiers include hydrogenated copolymers of styrene-butadiene, ethylene-propylene polymers, polyisobutenes, hydrogenated styrene-isoprene polymers, hydrogenated isoprene polymers, polymethacrylate acid esters, polyacrylate acid esters, polyalkyl styrenes, alkenyl aryl conjugated diene copolymers, polyolefins, polyalkylmethacrylates and esters of maleic anhydride-styrene copolymers. Dispersant viscosity modifiers (often referred to as DVM) include functionalised polyolefins, for example, ethylene-propylene copolymers that have been functionalized with the reaction product of maleic anhydride and an amine, a polymethacrylate functionalised with an amine, or styrene-maleic anhydride copolymers reacted with an amine.

Corrosion inhibitors include octylamine octanoate, condensation products of dodecenyl succinic acid or anhydride and a fatty acid such as oleic acid with a polyamine. Metal deactivators include derivatives of benzotriazoles, 1,2,4-triazoles, benzimidazoles, 2-alkyldithiobenzimidazoles or 2-alkyldithiobenzothiazoles. Foam inhibitors include copolymers of ethyl acrylate and 2-ethylhexylacrylate and optionally vinyl acetate. Demulsifiers include polyethylene glycols, polyethylene oxides, polypropylene oxides and (ethylene oxide-propylene oxide) polymers; pour point depressants including esters of maleic anhydride-styrene, polymethacrylates, polyacrylates or polyacrylamides. Seal swell agents include Exxon Necton-37™ (FN 1380) and Exxon Mineral Seal Oil (FN 3200).

Other performance additives include wax control polymers (including wax crystal modifiers and wax dispersants, such as ethylene vinyl acetate, fumarate vinyl acetate, copolymer esters or alkyl phenol resins), scale inhibitors including phosphate esters, gas-hydrate inhibitors (often known as freeze point depressant) including methanol or mixtures thereof. Typically, wax control polymers, scale inhibitors and gas-hydrate inhibitors may be used when the hydrocarbon fluid is a crude oil

### INDUSTRIAL APPLICATION

The composition of the invention is useful for providing at least one of acceptable low temperature performance properties, and/or a composition with a higher actives content (i.e. reduced amount of a solvent/carrier medium); and the reduction and/or inhibition of waxy deposits and/or gels in a subterranean oil reservoir, oil pipe line or storage vessel or other relevant equipment a hydrocarbon fluid, e.g., a crude oil may come in contact with. The composition of the invention is also useful in industrial and marine hydrocarbon fuel systems, including where fuel stream mixing may occur.

In another embodiment the invention is also useful for a liquid fuel for an internal combustion engine. In another embodiment the invention is useful for an oil of lubricating viscosity for lubricating an internal combustion engine.

The internal combustion engine includes a 2-stroke or 4-stroke engine fuelled with gasoline, diesel, natural gas or mixed gasoline/alcohol. The diesel engine includes both light duty and heavy duty diesel engines. The gasoline engine includes a direct injection gasoline engine.

Typically the amount of the hydrocarbyl- and hydroxy-substituted aromatic condensate present in a composition containing a liquid fuel and/or an oil of lubricating viscosity, in several embodiments ranges from 0.0001 wt % to 40 wt %, 0.1 wt % to 30 wt % or 1 wt % to 20 wt % of the composition.

Typically the amount of the hydrocarbyl- and hydroxy-substituted aromatic condensate present in a composition containing a crude oil in several embodiments ranges from 1 ppm to 20 wt %, or 5 ppm to 10 wt %, or 15 ppm to 3 wt %, or 20 ppm to 1 wt %. For example the hydrocarbyl- and hydroxy-substituted aromatic condensate may be present from 60 ppm to 500 ppm or 80 ppm to 350 ppm.

In one embodiment the composition and method provide acceptable low temperature properties in a hydrocarbon fluid without forming unacceptable amounts of waxy deposits and/or gels.

The following examples provide an illustration of the invention. These examples are non exhaustive and are not intended to limit the scope of the invention.

### EXAMPLES

### Preparative Example 1

### 82.5 mol % C24-C28 and 17.5 mol % C12 Alkyl Phenols

A reaction vessel is charged with 247.5 g of a mixture of a linear C₂₄-C₂₈ alkyl phenol and 52.5 g of branched iso-dodecyl phenol. The vessel is closed with a flange lid and clamped in place before adding nitrogen to form an inert atmosphere. The mixture is then heated to 85° C. and stirred for 30 minutes. A catalytic amount of sulphuric acid (2.6 g) is added dropwise through an equalizing dropping funnel. The contents of the reaction vessel are heated to 105° C. Paraformaldehyde (21.3 g) is added over a period of 35 minutes through a screw feeder. The contents of the reaction vessel are heated to 121° C. and held for 2 hours before adding 4.3 g of sodium hydroxide (in 50% solution) through a dropping funnel. The contents of the reaction vessel are heated to 150° C. and held for 2 hours. The contents of the reaction vessel are cooled to 76° C. before adding a diluent forming amount of an oil of lubricating viscosity and/or liquid fuel.

Preparative Example 2 is prepared by the same process as described for Preparative Example 1, except the amount of linear C₂₄-C₂₈ alkyl phenol and branched iso-dodecyl phenol is modified to produce a product with 90 mol % of C₂₄-C₂₈ alkyl phenol and 10 mol % of iso-dodecyl phenol.

Preparative Example 3 is prepared by the same process as described for Preparative Example 1, except the amount of linear C₂₄-C₂₈ alkyl phenol and branched iso-dodecyl phenol is modified to produce a product with 85 mol % of C₂₄-C₂₈ alkyl phenol and 15 mol % of iso-dodecyl phenol.

Preparative Example 4 is prepared by the same process as described for Preparative Example 1, except the amount of linear C₂₄-C₂₈ alkyl phenol and branched iso-dodecyl phenol is modified to produce a product with 80 mol % of C₂₄-C₂₈ alkyl phenol and 20 mol % of iso-dodecyl phenol.

Reference Example 1 is a commercially available C₂₄-C₂₈ alkyl phenol.

Reference Example 2 is a commercially available C₁₂-C₁₄ alkyl phenol.

### Test 1: Cycle Cooling Test

Samples containing 11.5 wt % and 15 wt % of the product of Preparative Examples 1 to 4 and Reference Examples 1 and 2 are heated in an oven at 50° C. The samples are then cooled for 12 hours at −5° C. and −10° C. The samples are then rated as being solid, liquid or gelatinous. The samples are warmed to ambient temperature over a period of 2 hours and frozen up to 3 times more. Acceptable results are obtained for samples that remain liquid at −5° C. or −10° C. after a third freeze cycle.

The results obtained for compositions containing the product of Preparative Examples 1 to 4 at treat rates of 11.5 wt % and 15 wt %; and at −5° C. and −10° C. all remain liquid after the third freeze cycle. Reference Examples 1 and 2 remain liquid at −5° C. after the third freeze cycle; but become solid or gelatinous after the second freeze cycle at −10° C. Hence the results of the cycle cooling test indicate that the composition of the invention provides acceptable performance after the third freeze cycle whereas Reference Examples fail the test.

In summary, the composition and method of the invention provide acceptable low temperature properties in a hydrocarbon fluid without forming unacceptable amounts of waxy deposits and/or gels.

It is known that some of the materials described above may interact in the final formulation, so that the components of the final formulation may be different from those that are initially added. The products formed thereby, including the products formed upon employing the composition of the present invention in its intended use, may not be susceptible of easy description. Nevertheless, all such modifications and reaction products are included within the scope of the present invention; the present invention encompasses the composition prepared by admixing the components described above.

Each of the documents referred to above is incorporated herein by reference. Except in the Examples, or where otherwise explicitly indicated, all numerical quantities in this description specifying amounts of materials, reaction conditions, molecular weights, number of carbon atoms, and the like, are to be understood as modified by the word “about.” Unless otherwise indicated, each chemical or composition referred to herein should be interpreted as being a commercial grade material which may contain the isomers, by-products, derivatives, and other such materials which are normally understood to be present in the commercial grade. It is to be understood that the upper and lower amount, range, and ratio limits set forth herein may be independently combined. Similarly, the ranges and amounts for each element of the invention may be used together with ranges or amounts for any of the other elements. As used herein any member of a genus (or list) may be excluded from the claims.

As used herein, the term “hydrocarbyl substituent” or “hydrocarbyl group” is used in its ordinary sense, which is well-known to those skilled in the art. Specifically, it refers to a group having a carbon atom directly attached to the remainder of the molecule and having predominantly hydrocarbon character. Examples of hydrocarbyl groups include:

(i) hydrocarbon substituents, that is, aliphatic (e.g., alkyl or alkenyl), alicyclic (e.g., cycloalkyl, cycloalkenyl) substituents, and aromatic-, aliphatic-, and alicyclic-substituted aromatic substituents, as well as cyclic substituents wherein the ring is completed through another portion of the molecule (e.g., two substituents together form a ring);

(ii) substituted hydrocarbon substituents, that is, substituents containing non-hydrocarbon groups which, in the context of this invention, do not alter the predominantly hydrocarbon nature of the substituent (e.g., halo (especially chloro and fluoro), hydroxy, alkoxy, mercapto, alkylmercapto, nitro, nitroso, and sulfoxy);

(iii) hetero substituents, that is, substituents which, while having a predominantly hydrocarbon character, in the context of this invention, contain other than carbon in a ring or chain otherwise composed of carbon atoms. Heteroatoms include sulfur, oxygen, nitrogen, and encompass substituents as pyridyl, furyl, thienyl and imidazolyl. In general, no more than two, or no more than one, non-hydrocarbon substituent will be present for every ten carbon atoms in the hydrocarbyl group; typically, there will be no non-hydrocarbon substituents in the hydrocarbyl group.

While the invention has been explained, it is to be understood that various modifications thereof will become apparent to those skilled in the art upon reading the specification. Therefore, it is to be understood that the invention disclosed herein is intended to cover such modifications as fall within the scope of the appended claims.

What is claimed is:

1. A composition comprising:
(a) a hydrocarbon fluid; and
(b) a hydrocarbyl- and hydroxy-substituted aromatic condensate comprising the reaction product of:
(1) at least about 75 mole percent based on the total amount of (b)(1) and (b)(2) of a hydrocarbyl- and hydroxy-substituted aromatic compound wherein the hydrocarbyl substituent is a linear substituent containing about 8 to about 36 carbon atoms;
(2) about 0.001 to about 25 mole percent based on the total amount of (b)(1) and (b)(2) of a hydrocarbyl- and hydroxy-substituted aromatic compound wherein the hydrocarbyl substituent is a branched substituent containing about 8 to about 20 carbon atoms; and
(3) an amount of an aldehyde, ketone, or reactive equivalent thereof effective to condense (1) and (2) to form the hydrocarbyl- and hydroxy-substituted aromatic condensate.

2. The composition of claim 1, wherein the hydrocarbyl- and hydroxy-substituted aromatic compound of (b)(1) has a linear hydrocarbyl substituent containing about 20 to about 30 carbon atoms.

3. The composition of claim 2, wherein the hydrocarbyl- and hydroxy-substituted aromatic compound of (b)(1) has a linear hydrocarbyl substituent containing about 24 to about 28 carbon atoms.

4. The composition of claim 1, wherein the hydrocarbyl- and hydroxy-substituted aromatic compound of (b)(1) is present at about 75 mol % to about 95 mol % based on the total amount of (1) and (2).

5. The composition of claim 1, wherein the hydrocarbyl- and hydroxy-substituted aromatic compound of (b)(2) has a branched hydrocarbyl substituent containing about 10 to about 15 carbon atoms.

6. The composition of claim 1, wherein the hydrocarbyl- and hydroxy-substituted aromatic compound of (b)(2) is present at about 5 mol % to about 25 mol % based on the total amount of (1) and (2).

7. The composition of claim 1, wherein the aldehyde is formaldehyde formalin, paraformaldehyde or mixtures thereof.

8. The composition of claim 1, wherein the hydrocarbyl- and hydroxy-substituted aromatic condensate has a number average molecular weight of about 3000 to about 25,000.

9. The composition of claim 1, wherein the hydrocarbyl- and hydroxy-substituted aromatic condensate is represented by the formula: 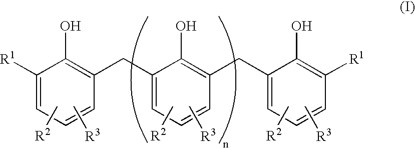
wherein
n is an integer from 0 to about 10;
R¹ is hydrogen or alkyl with 1 to about 4 carbon atoms;
R² is hydrogen or a linear alkyl or linear alkoxy group containing about 24 to about 28 carbon atoms;
R³ is hydrogen or a branched alkyl group containing about 12 to about 14 carbon atoms; with the proviso that when R² is hydrogen, R³ is the branched alkyl group; and when R³ is hydrogen, R² is the linear alkyl or linear alkoxy group,
wherein the hydrocarbyl- and hydroxy-substituted aromatic condensate comprises a hydrocarbyl-R² group substituted- and hydroxy-substituted aromatic compound which is present in an amount from about 75 mol % to about 95 mol % based on the total amount of (b)(1) and (b)(2); and a hydrocarbyl-R³ group substituted- and hydroxy-substituted aromatic compound which is present in an amount from about 5 mol % to about 25 mol % based on the total amount of (b)(1) and (b)(2).

10. The composition of claim 9, wherein when R² and R³ in the para position relative to the hydroxyl group when R² and R³ are the alkyl or alkoxy groups.

11. The composition of claim 1, wherein the hydrocarbon fluid is a crude oil.

12. The composition of claim 1, wherein the hydrocarbon fluid is a liquid fuel or an oil of lubricating viscosity.

13. A method of controlling waxy deposit and/or gel formation in a hydrocarbon fluid, the method comprising mixing:
(a) a hydrocarbon fluid; with
(b) a hydrocarbyl- and hydroxy-substituted aromatic condensate comprising the reaction product of:
(1) at least about 75 mole percent based on the total amount of (b)(1) and (b)(2) of a hydrocarbyl- and hydroxy-substituted aromatic compound wherein the hydrocarbyl substituent is a linear substituent containing about 8 to about 36 carbon atoms;
(2) about 0.001 to about 25 mole percent based on the total amount of (b)(1) and (b)(2) of a hydrocarbyl- and hydroxy-substituted aromatic compound wherein the hydrocarbyl substituent is a branched substituent containing about 8 to about 20 carbon atoms; and
(3) an amount of an aldehyde, ketone, or reactive equivalent thereof effective to condense (1) and (2) to form the hydrocarbyl- and hydroxy-substituted aromatic condensate.
